(19) United States
(12) Patent Application Publication
(10) Pub. No.: US 2005/0053904 A1
(43) Pub. Date: Mar. 10, 2005

(54) System and method for on-site cognitive efficacy assessment

(76) Inventors: Jennifer Shephard, Watertown, MA (US); Stephen Kosslyn, Cambridge, MA (US)

Correspondence Address: ROBERTS ABOKHAIR & MARDULA, SUITE 1000, 11800 SUNRISE VALLEY DRIVE, RESTON, VA, 20191, US

(21) Appl. No.: 10/918,592

(22) Filed: Aug. 13, 2004

Related U.S. Application Data
(60) Provisional application No. 60/494,883, filed on Aug. 13, 2003.

(51) Int. Cl. G09B 19/00; A61B 5/00

(52) U.S. Cl. 434/236; 600/300; 128/897; 128/920

(57) ABSTRACT

A system and method of determining diminishment in brain function in relation to at least one of specific adverse factors and specific tasks. The system involves establishing threshold levels for at least one brain function that is either affected by specific adverse factors or is used to perform specific tasks, or both. A test-taker is given a battery of tests from a portable testing unit, where the tests have been tailored to measure specific cognitive functions that can be affected by adverse factors, cognitive functions that are required to perform specific tasks, or both. Scores from the tests are compared to the threshold levels required to perform a task, and a determination is made concerning the capability of the individual to handle the task(s) in question. The portable testing unit may be connected to equipment whose use is denied for test results that fall below a normative level for the activity.

### CROSS REFERENCE TO RELATED APPLICATIONS

This application claims priority under 35 U.S.C. § 119(e) from provisional application No. 60/494,883 filed Aug. 13, 2003. The 60/494,883 provisional application is incorporated by reference herein, in its entirety, for all purposes.

### BACKGROUND

Human error cuts across multiple domains (transportation accidents, mistakes in medical treatment, loss of computerized data) and results in an incalculable loss of productivity—and even loss of human life—every year. For example, in the Navy alone, certain studies in 2000 note that a reduction by half of aviation mishaps due to human error would save 250 lives and $1 billion in a period of five years. A more recent estimate (2002) notes the financial cost at $4.3 billion over five years, with an additional $20 to $30 billion in indirect costs such as litigation, investigation and program delays. Safety officials in the Navy claim that human error is responsible for up to 85% of aviation mishaps, that this has not changed much over the last 25 years—and that this will be ‘the toughest nut to crack’. Further, lost profits to American businesses due to shift work fatigue in the U.S. total about $206 billion (2003 estimate). This breaks down to $169 billion due to employee turnover, absenteeism, and reduced productivity, $28.2 billion in increased healthcare costs, and $8.5 billion due to job-related accidents. Surgeons, pilots, air traffic controllers, emergency-crew workers, and truck-drivers, among others, would benefit from a quick cognitive check that would potentially eliminate or reduce the incidents of fatigue related mishaps and mistakes.

Identifying what parts of the brain are responsible for the performance of specific tasks, functions and other processes has been the focus of cognitive psychology and cognitive neuroscience. Parts of the brain that, for example, control speech, motor skills, perception, and some forms of memory and reasoning have been largely mapped.

It has also been recognized that brain function is significantly affected by many factors, including sleep deprivation, drugs and alcohol, and sensory distractions, such as temperature, noise, light, pain, etc. An immense number of factors can adversely affect brain performance.

Specific cognitive abilities are summarized herein for clarity. For instance, three types of attention (vigilance, filtering, and divided) have been characterized in detail. If a person is unable to allocate attention appropriately, he or she is not likely to perform well in general. Vigilance is the ability to concentrate and wait for a specific event over a sustained period of time. For example, one test of vigilance presents a series of small geometric shapes one at a time on the screen. The set of shapes contains one target (a particular parallelogram) and five distracters. Because targets are presented occasionally and randomly, the test-taker must maintain concentration to detect them. Filtering is the ability to focus on what is important and ignore what is irrelevant to the task at hand. The classic test of filtering is the Stroop (b 1935) task, in which participants see color names printed in different color inks (e.g., the word “red” might be printed in blue ink), and are required to name the color of the ink while ignoring the meaning of the word. Divided attention requires one to focus on two unrelated types of stimuli or stimulus features. In one version, a series of geometric shapes (circles, triangles, squares, and stars) in different colors (red, blue, green, purple) or shades (depending on whether the color or monochromatic version is used) are presented on the screen, one at a time. Test-takers are required to press one button if the shape is a triangle or is red (or dark grey, in the monochromatic version) in color and another button if the shape is a circle or blue (light grey) in color (no triangles are blue/light grey and no circles are red/dark grey, to prevent response competition).

Another cognitive function is working memory. Information often must be “held in mind” as one manipulates it in some fashion, which requires working memory. Difficulty in using working memory interferes with thinking and problem solving, and most people who suffer from a working memory deficit are unable to think through and diagnose the cause of their difficulty because of the nature of the problem itself. A now-classic working memory task requires participants to see a series of stimuli and respond when a stimulus appears that had also appeared one, two (or, in more challenging versions, three) trials earlier in the sequence. A verbal version of this task requires the test-taker to keep track of a sequence of digits and a spatial version requires the test-taker to keep track of a sequence of locations.

Another cognitive function is motor control. It's important to discover whether changes in response times or errors on other tests may be due simply to a slower motor response rather than other cognitive deficits. For instance, hunger, cold, or drug withdrawal tremors could affect the ability of a test-taker to move his/her fingers quickly and accurately across the rather small and closely-spaced keys on many portable testing devices.

Still another cognitive function is problem solving. To assess disruptions of very “high order” cognitive abilities two tests have been prepared, which require different types of problem solving and reasoning. The first is verbal problem solving. The classic three-term series reasoning problems, such as “Kate is not as young as Sam; Kate is less old than Thomas. True or false: Sam is not oldest,” are administered. Test takers indicate their responses by pressing one button for true and another for false. The second is spatial problem solving. Mental rotation problems are administered. Pairs of stimuli are presented, with one member of the pair rotated relative to the other. Test-takers are required to try to “mentally align” one form so that it is at the same angle as another, and then to decide whether the two are identical or mirror-reversals.

Yet another cognitive function is cognitive set switching. The classic tests of cognitive switching are the Wisconsin Card Sort test and the Odd-Man-Out test. These tasks require participants to sort stimuli (typically simple figures or letters) according to different criteria. The stimuli are presented on a deck of cards, and the figures or letters on each card can be classified into more than one category (e.g., a given card might have three purple squares; this card could be categorized according to number, color, or shape). In the Wisconsin Card Sort test, for instance, participants begin to sort cards into different piles based on one of three possible criteria (shape, color, number). They receive feedback after each trial (they are simply told whether they have sorted that particular stimulus correctly), and from this feedback must learn the appropriate sort criterion (predetermined by the administrator). Once they have learned the criterion (learning is defined as correctly responding to a specific number of trials in a row), the sort criterion is arbitrarily switched (e.g., from number to shape), and the process begins again. The key measure is how many trials a participant requires before he or she discovers the new criterion. A simplified version of the Odd-Man-Out test has been implemented, in which on each trial a series of four letters (e.g., T H t H) appears, and the test-taker must decide which letter does not belong in the series (in this case, the lowercase ‘t’). There are two stimulus criteria (letter identity and capitalization), only one of which applies to any given trial. Two, three, four or five (randomly determined) trials in a row have the same criterion, and then the criterion switches. Response time (RT) and error rates (ER) are measured when the appropriate stimulus dimension has been switched. Test-takers will form a mental set based on each series of trials they receive, and thus increases in RT should be found, and possibly decrements in accuracy, when the criterion is shifted.

Though many of the cognitive processes underlying these tests have been identified and their neural underpinnings mapped, there lacks a system for determining at what point a person's brain functions are below a level that would be appropriate to perform certain real-world—often job-related—tasks (hereafter referred to simply as “task[s]”). Moreover, present attempts to monitor comparative levels of brain function are obtrusive and have not been able to be site-located. In other words, a truck driver may be accurately diagnosed with sleep deprivation at a clinic, but there is no system in place for determining, on the job, whether a truck driver is sleep-deprived, and whether this is affecting the cognitive abilities required for the tasks that need to be performed in driving a truck.

Such methodologies and techniques are increasingly needed in the military, as well as many other walks of life. Human error cuts across multiple domains and results in an incalculable loss of productivity and even human life every year. What is needed is a system for identifying threshold levels of cognitive performance required for specific tasks. Such a system should be portable and can be used on location as needed.

### SUMMARY

This system and method of the present invention provides an “early warning,” indicating when a person is suffering from stress-related deficits that may affect performance of a particular activity (truck driver, astronaut, surgeon and others). The results can be used to warn a person to pay additional attention or take a countermeasure (even if only a brief rest). The present invention comprises software for administering cognitive tests on a handheld device and providing immediate user feedback, as well as desktop software for authoring new tests and scoring results in detail. Using the present invention the effectiveness of countermeasures, such as the utility of caffeine and exercise to counteract fatigue or relaxation techniques to cope with noise and performance anxiety can be achieved.

The present invention provides a noninvasive, highly portable, quickly administered system and method of determining diminishment in brain function in relation to at least one specific adverse factor and/or specific task. A test-taker is given a battery of tests from a portable testing unit, where the tests have been tailored to measure specific cognitive functions. The functions selected have previously been shown to rely on specific brain systems. The particular tests that are administered may depend on the nature of the task to be performed and the nature of the adverse stimuli. Different combinations of cognitive functions, which are assessed by different tests, underlie different tasks and are affected by different adverse factors. Results from the tests are automatically calculated and compared to normative data, and the scores and normative data are displayed, showing the test-taker's relative “performance level” at the current time, and allowing one to predict whether that individual will be capable of handling the task(s) in question.

Software of the present invention presents cognitive tests on a handheld device while simultaneously processing the user's data in real time to present immediate feedback. This software allows test results to be obtained and is flexible, allowing new tests to be created easily. A neurologically valid battery of cognitive tests is programmed into the present invention and stored in the memory of a handheld device (although this format is not meant as a limitation). This battery assesses a wide range of cognitive abilities. Based on the psychological and neuroscience literature, the present invention has been equipped with an initial set of nine tasks that tap basic information processing in the brain (for instance, attention, working memory, and problem-solving). Further, the present invention may be used to evaluate the effectiveness of various countermeasures (for example, drugs or exercise to manage fatigue, relaxation techniques to manage noise-induced stress) so that a user can select the best method to use to over come any cognitive shortfall.

An embodiment of the present invention uses a series of tests to evaluate a test-taker's (or “user's”) current cognitive functioning in several domains. Cognitive abilities are affected by adverse factors (noted above), or are necessary for performing various “real world” tasks (hereafter simply referred to as “tasks”), or both. Because an aspect of the present invention is that such tests are to be portable and easily administered, the tests have been implemented on a portable device, namely a Palm-based PDA (although this is not meant as a limitation). The tests comprise images or sounds (or a combination thereof), and require user responses. In an embodiment of the present invention, test results are expressed as “scores” comprising measures of an error rate and a response time.

Certain physiological functions may also be affected by adverse factors and thus may also serve as reliable predictors of task performance. Portable devices for measuring physiological response of humans are known in the art and when used in conjunction with cognitive measurements are considered to be within the scope of the present inventions as well.

Installed on the portable device is software for conducting the tests, for automatically recording time and date stamps and response times each time a user completes a test, and for storing user-entered data. As noted above, software for collecting and storing the physiological measurements is also a part of the present invention. Normative data for the tests (and for the physiological measurements as well) is stored on the device. Finally, installed on the portable device are algorithms for analyzing the user data and for comparing the user “scores” with normative data to determine a performance level for a test-taker.

In an embodiment of the present invention, algorithms are used to evaluate a user's performance level with respect to thresholds and to make recommendations regarding the test-taker's ability to perform a task. In one embodiment of the present invention, the algorithms are installed on the portable device. Alternatively, the algorithms are installed on another computer and the user's performance level is provided to that computer for evaluation. Part of the present invention is software that is installed on a desktop computer for the purpose of creating new tests (a compiler that converts html scripts to a format appropriate for the handheld program) that can ultimately be downloaded to a PDA or to an embedded program associated with the equipment that is to be operated. User-entered data comprises a reference number, year of birth, years of education, handedness, gender, and responses to test trials. Additionally users can input a short note before they take any of the tests, for example, “didn't sleep well last night”, or any other information that might be useful to an evaluator of the user's performance. It is therefore an aspect of the present invention to provide a portable testing system to evaluate brain function.

It is another aspect of the present invention to assess different cognitive functions and to relate cognitive functions to a task to be performed by a test-taker.

It is still another aspect of the present invention to express the test results as “scores” that are measures of an error rate and a response time.

It is another aspect of the present invention to determine a test-taker's performance level by comparing the test-taker's scores to a normative data for a particular test.

It is yet another aspect of the present invention to determine a test-taker's ability to perform a particular task by administering a test and comparing the test-taker's performance level to a pre-established threshold.

It is still another aspect of the present invention to use test results to permit or deny access to equipment associated with an activity if test results fall below a normative level.

In an embodiment of the present invention, a user completes one or more tests on the portable device. The “data” (or “raw data”)—responses and response times (RTs)—for a specific test results in a series of “scores.” Such score may be (without limitation) ‘mean RT’, ‘RT variance’, and ‘error rate’ (ER). Both the raw data and the scores are stored and may be uploaded to another computer. However, the scores (but not the raw data) may also be displayed on the portable device if an authorization code is entered.

When the scores are displayed on the portable device they are presented both numerically and overlaying a histogram of normative data for a particular test. (Each test has its own histogram.) The normative data may be from similar users or may consist of the user's own past performance scores for a particular test.

The results comprising the scores are absolute numbers, e.g., a mean RT of 950 milliseconds (ms) or an ER of 13%. A “performance level,” however, is a relative number, and compares the test-taker's scores with normative data. Performance level may be expressed (or displayed on the histogram) for example as “1.5 standard deviations above the mean.”

The tests implemented on the present invention measure cognitive impairment due to at least one adverse factor, or predict performance on at least one specific task, or both. Based upon prior testing, a specific “threshold” (measured in terms of a score value or performance level value) required for satisfactory task performance is determined. A threshold is associated with a degree of predictive accuracy that the user will be able to perform a specific task. For example, a threshold may be defined as “a performance level that provides 95% confidence, plus or minus one sigma, that task X may be performed successfully.” The accuracy imposed by the threshold may vary depending on the task to be performed. For example, 95% accuracy for “stamp licking” may be more than satisfactory, but a higher threshold may be required for operating a nuclear reactor.

A threshold may comprise absolute numbers (i.e., “scores”, for instance an ER of 15%), but may also be relative (i.e., “performance levels”, for instance an ER of 1 standard deviation greater than the user's own normal ER). Depending on the task, one or more thresholds may be appropriate. For instance, for certain test/task combinations, there may be one threshold, performance above which predicts satisfactory task performance and performance below which predicts unsatisfactory task performance. In other cases two thresholds, for instance, may be more appropriate (performance below the low threshold would predict failure on the task, performance above the high threshold would predict success, and performance between the two thresholds would predict successful task performance *only if* certain precautionary measures are taken).

According to an embodiment of the present invention, an “efficacy level” characterizes the test-taker's performance with respect to an appropriate set of thresholds, and may be expressed in qualitative terms such as “poor,” “satisfactory,” “excellent,” etc.

Based on prior testing, a determination of what, if any, precautionary measures can be taken to reduce the impairment caused by one or more adverse factors and thus boost performance on one or more tasks can be made. Using the thresholds and the knowledge gained from data mining of scores over an extended period of time and users, a “recommendation” can be offered to the test-taker or the test-taker's supervisor, given specific test scores. The recommendations may comprise without limitation, (a) a warning that test performance is so low that the user is likely to fail at performing a given task and thus should not attempt it, (b) advice that test performance is impaired but that various precautionary measures (to be specified depending on the adverse factor, the task to be completed, or a combination thereof) may reduce impairment and increase the likelihood that task performance will be successful, or (c) a suggestion to perform the task because no impairments are present.

It should be noted that physiological measurements and thresholds may also play a role in the recommendations that are made. Further, composite sets of responses will also be analyzed to determine the covariance associated with these various responses to allow greater predictability in the performance of certain tasks. An embodiment of the present invention provides a method for assessing relative levels of cognitive performance. A test-taker is provided a test object indicative of a cognitive function. The test-taker provides a response from which a score is calculated. In one embodiment of the present invention the response comprises receiving an answer and a measure of response time. The score is evaluated against normative data to determine a performance level of the cognitive function of the test-taker. In an embodiment of the present invention, the performance level relates to at least one of attention, working memory, problem-solving, cognitive-set-switching, perceptual control, motor control, and cognitive focus. The score and/or the performance level are displayed. Displaying the score and the performance level may require an authorization code.

A time and date stamp may be associated with the response, the score and/or the performance level. Additionally, personal data of the test-taker may be associated with the response, the score and/or the performance level. In an embodiment of the present invention, personal data comprises at least one of year of birth, gender, handedness, years of education, and reference number. Further, the user may enter short notes that may affect the interpretation of test results such as “Did not sleep well last night”; “I am taking medication of a particular type.”

In another embodiment of the present invention, the method described above is practiced on a portable testing unit. The response, the score and the performance level may be stored on the portable testing unit. Additionally, the score is evaluated on the portable testing unit. Alternatively, the score is evaluated on a second device that receives the score from the portable testing unit. The score and/or the performance level may be displayed on the portable testing unit or on a second device.

Another embodiment of the present invention provides a method for determining impairment in brain function in relation to specific adverse factors. A threshold for cognitive performance for an adverse factor is established. In an embodiment of the present invention, an adverse factor comprises sleep deprivation, drugs, alcohol, sensory distractions, physiological conditions, and psychological conditions. A test tailored to measure impairment due to the adverse factor is administered to a test-taker on a portable testing unit, such as a portable computer, to produce a score. The test-taker may be an individual or a group of persons taking the test individually. An efficacy level of the test-taker is determined by comparing the score to the threshold. In another embodiment of the present invention, the test further comprises at least one of a physiological measurement, an auditory component, a visual component, and a reflex component.

### DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 illustrates a block diagram of a logical flow of an assessment process according to an embodiment of the present invention.

FIG. 2 illustrates a portable test unit according to an embodiment of the present invention.

FIG. 3 illustrates the initial screens a user would see on the portable unit according to an embodiment of the present invention.

FIG. 4 illustrates a sample test “instructions screen” that may be displayed to the user once the test function has been selected according to an embodiment of the present invention.

FIG. 5 illustrates a display of test results according to an embodiment of the present invention.

### DETAILED DESCRIPTION

For the sake of clarity, the follow terms have the meaning ascribed to them:
adverse factor—a factor that interferes with cognitive function.
behavioral measure—a measure of the physical response(s) to a test and the accuracy of the response(s), for example, the response time(s), and error rate.
cognitive focus—a test that taps vigilance and verbal and spatial working memory together.
cognitive function—the mental processes by which information is absorbed and reacted to, as by awareness, perception, reasoning, and judgment.
efficacy level—a qualitative expression of the performance of a test-taker relative to a threshold.
normative data—a body of data acquired from testing a particular test-taker or from a population of test-takers.
performance level—a measure of performance of a test-taker to a test relative to normative data.
physiological measure—a measure indicative of the state of the body or bodily functions.
recommendation—a conclusion reached after assessment of the score and/or performance level of a test-taker.
score—measures of performance of a test-taker for a particular test stated in absolute terms.
test-taker—a person taking a test or a set of tests.
threshold—a condition based on a score value or a performance level value.

The present invention comprises three components: a compiler that translates html scripts into tests that run on a handheld device; a PDA component that collects information from the user, presents the tests, stores data, and displays results; and battery of psychological tests. Currently the present invention is configured to run on the Palm OS™; the compiler runs on both Macintosh and Windows operating systems.

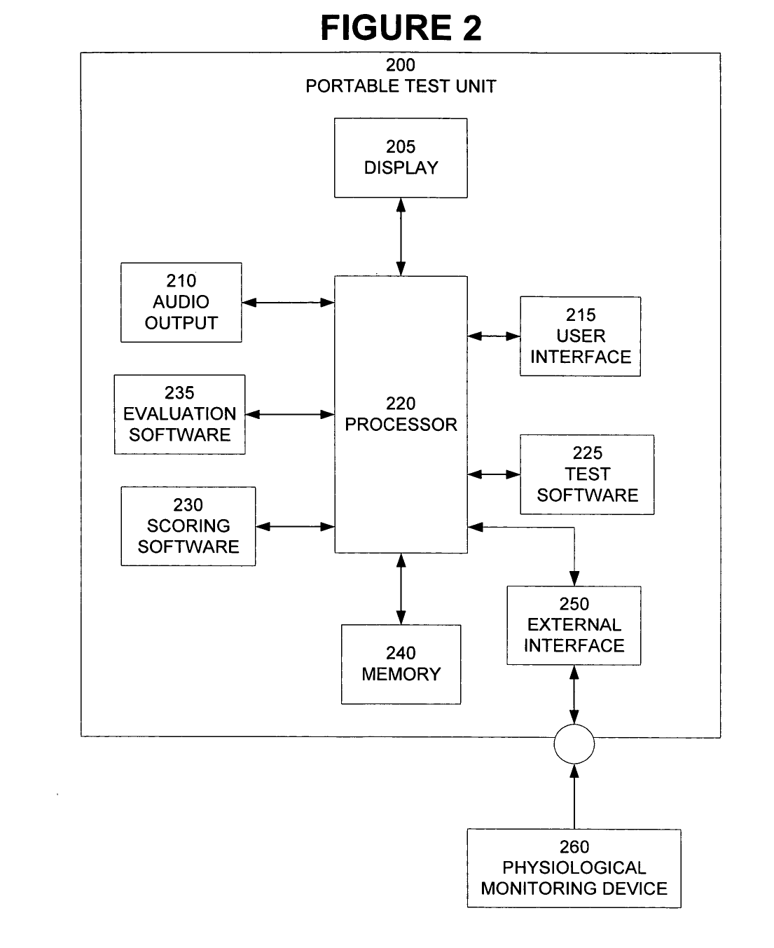

The present invention is designed to read both text and picture stimulus files, present stimuli for specific amounts of time with a variable inter-stimulus delay, provide auditory feedback after incorrect responses to practice trials, and record responses (made by pressing one of up to six different keys) and response times. The responses are stored, along with a user ID and basic demographics (year of birth, years of education, sex, and handedness; see FIG. 2, left) and time-and-date stamp. Users may see a display of their results immediately after each test; the results are presented numerically and as histograms.

The histograms compare a user's current errors, response times, and response time variance to the performance of all other users, other users of the same sex as the current user, and to the user's own past scores (this last requires the user to perform the task a number of times under standard conditions in order to generate a normal histogram). Complete data sets (with user information and responses and response times for each trial of each administration of each task) are stored on the PDA for later upload to a desktop computer. The datasets are stored as pdb files, which can be opened in a spreadsheet program for more detailed analysis. It should be noted that, while a PDA, and associated operating systems and data formats are disclosed herein, this is not meant as a limitation. Other systems having computational capabilities in the form of chips sets, user interactive means such as keyboards, touch screens, voice input and the like are equally suitable for the present invention. For example, using current cellular telephone capabilities, programs of the present invention can be downloaded and tests administered on a cell-phone type platform as well.

The present invention is flexible, allowing one to program new tasks quickly and easily as the need arises (e.g., to test a specific aspect of functioning that is needed for a particular real-world application). The tasks differ only in the instructions (which are presented in initial screens), stimuli, and the number of response keys used. The tasks have been designed in such a way that memorizing the stimuli does not give one an advantage in responding, and stimuli are presented in different random orders each time the task is performed, so that the order of answers cannot be memorized. Thus, even if users encounter the same items frequently within a short period of repeated testing, they will not be able to learn the correct responses by rote. There is also an option to “load in” more stimuli than are needed, and stimuli are selected from this pool randomly each time the test is performed (and then removed from the pool until all the stimuli have been used), thus minimizing the number of times any given stimulus is repeated.

Embodiments of the present invention provide a system and method of determining impairment to brain function in relation to specific adverse factors, specific tasks, or both. Different adverse factors may have cumulative, but not necessarily compound effects on brain functions. For example, after two drinks, efficacy in performing a specific task may be reduced by 30%. With loud noises and flashing lights, efficacy may be reduced by 20%. If all these adverse factors are in play, however, efficacy may be reduced by only 35% or by as much as 80%. Different adverse factors not only affect brain function in different ways, but their cumulative effects can have surprising consequences. Furthermore, the type of task being performed may suffer more or less depending on the adverse factor and what specific cognitive functions are affected by that factor. Therefore determining the efficacy of a test-taker is predicated both on the adverse factors and on the task to be performed, and the tests may be adapted accordingly.

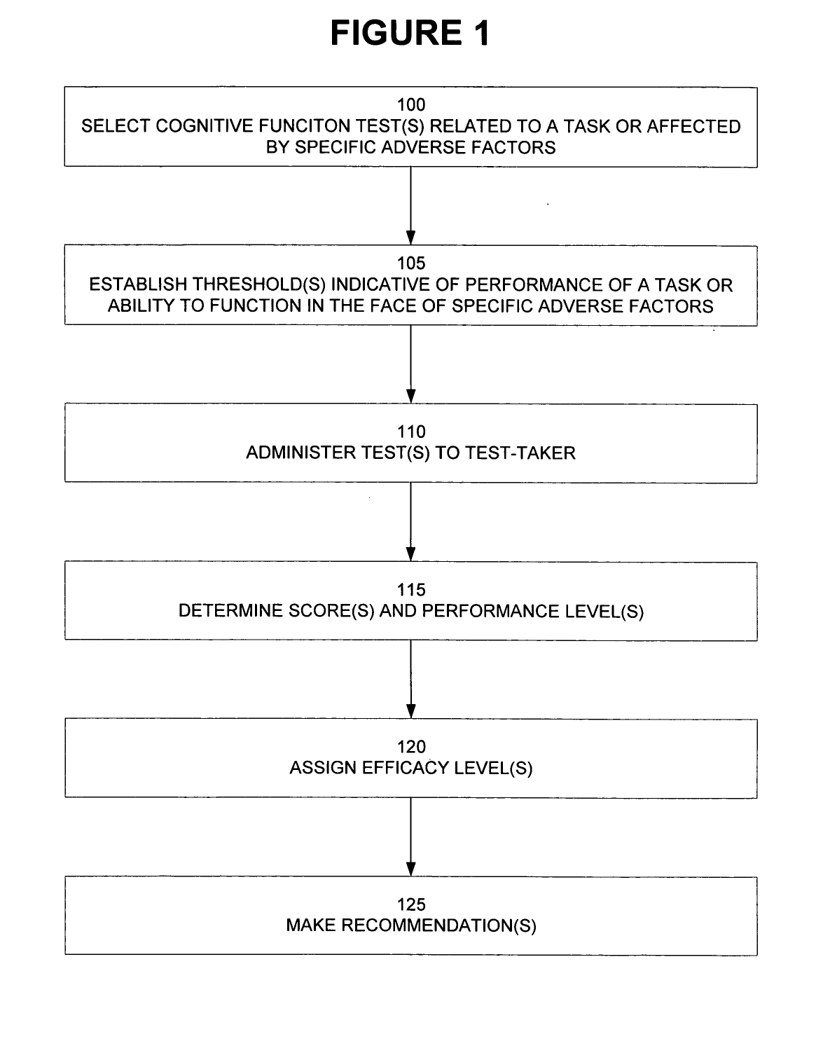

FIG. 1 illustrates a block diagram of a logical flow of an assessment process according to an embodiment of the present invention. Cognitive function tests related to a task or that are affected by specific adverse factors are selected 100. A test may be tailored to measure the functioning of specific brain systems indicative of the cognitive functions used in a specific task, but the test itself does not necessarily have to simulate that task. Performing a seemingly dissimilar test can therefore give a thorough assessment of the parts of the brain used in performing the specific task. Examples of specific tasks are operating a motor vehicle, piloting an aeronautical vehicle, piloting a marine vessel, operating machinery, performing diagnostic procedures, performing tasks requiring good hand-eye coordination (including those used in performing surgery), and assessing environmental situations. The test(s) may be tailored to specific tasks, and new tests may be added as required by the test-taker or those monitoring the test-taker.

The performance of mundane and obvious tasks, such as cooking, or playing sports, may also be evaluated. It is the nature of adverse factors that they affect self-diagnosis as well as other performance factors, so that even mundane tasks may be affected by impaired brain function and the individual so affected may not realize how poor his/her performance may be and why. Examples of specific adverse factors are sleep deprivation, drugs, alcohol, sensory distractions (such as hunger, noise, light, heat, cold, pain, etc.), physiological conditions (e.g., injury, illness, malnutrition, hypoxia, etc.) and psychological conditions or states (such as depression, fear, anxiety, preoccupation, etc.). The number of adverse factors involved are numerous and the present invention is not limited to those mentioned. The tests may be modified to group certain adverse factors together, such as all sensory distractions or all physiological conditions, making a general determination on the group. Some adverse factors may be more user-specific, such as attention deficit disorder, concussion, dyslexia, hypoglycemia, and others.

One or more thresholds indicative of performance of the task or performance in the face of specific adverse functions are established 105. In embodiments of the present invention, relative levels of performance of specific cognitive and/or physiological functions, in the presence of specific adverse factors, are established as predictors of satisfactory performance of specific tasks. A “threshold” may be calculated by measuring groups of similar individuals performing similar tests under similar adverse conditions, or prior to performing similar real-world tasks, or by other methods known in the art. A set of thresholds, as used herein, may refer to a single threshold (performance below which predicts failure to perform a specific task and above which predicts successful performance of a specific task), or as many thresholds as required. By way of illustration and not as a limitation, a set of thresholds may comprise two thresholds, where performance below the lower threshold would predict the test-taker's failure to perform a task successfully, where performance between the thresholds might predict the test-taker's success at a task if precautionary measures are taken, and where performance above the higher threshold would predict the test-taker's successful performance of a task without further intervention. As will be appreciated by those skilled in the art, other combinations of thresholds may be used without departing from the scope of the present invention.

A test is administered to a test-taker 110. Tests may be given to individuals or groups of individual test takers. Thus, a test may determine the overall functionality of a group, such as the overall alertness. Everyone in the group may be given the same test (separately), or specific individuals in the group may be given modified tests. For example, the efficacy of a leader is generally more important than the efficacy of one of a hundred subordinates. The tests may reflect this, so that the scores can be weighted to give the overall level of efficacy of the group (as measured by the scores or performance achieved by a control group of individual test takers), and predictions about the group's performance on other tasks can be made accordingly.

Scores and performance levels are determined 115. A score is an absolute measure of performance stated in terms of error rate or response time (mean RT, RT variance, ER, or various measurements of physiological functioning). A performance level is a relative measure determined by comparing a score value to normative data for a particular test. The normative data may be obtained from test results across similar population of test-takers or against the test-taker's own past performance. A “performance level” and may be expressed in terms of deviation from the mean (e.g., “one standard deviation above the mean RT”).

Based on how a test-taker performs compared to one or more threshold levels an “efficacy level” is determined 120, and “recommendations” made 125. In an embodiment of the present invention, an efficacy level is a subjective measure of performance relative to a threshold, e.g., “poor,” “satisfactory,” “excellent,” etc. A recommendation may warn that a task should not be performed, or advise that extra caution or precautions be taken, or suggest that additional tests be taken again within a certain time frame.

In an exemplary embodiment of the present invention, a set of tests designed for field use is used to unobtrusively and non-invasively assess the cognitive state of individuals and teams, including readiness for specific tasks. In another embodiment of the present invention, test conditions comprise adverse circumstances (such as sleep-deprivation); the cognitive readiness of individuals and teams is then predicted.

FIG. 2 illustrates a portable test unit according to an embodiment of the present invention. A processor 220 operates test software 225, scoring software 230, and evaluation software 235. Processor 220 receives input from a test-taker (not illustrated) via user interface 215. Visual information is conveyed to the test-taker by display 205 and audio information is provided to the test-taker by audio output 210. In an embodiment of the present invention, display 205 and user interface 215 are integrated into a touch-sensitive screen. Memory 240 receives and stores test data generated by a test-taker during the administration of a test using test software, scores generated by the scoring software 230, and normative data and threshold values used by evaluation software 235 to evaluate a test-taker's test results. In an alternate embodiment, evaluation software 235 also determines the test-taker's efficacy level and makes recommendations accordingly.

Processor 220 also connects to external interface 250. As illustrated in FIG. 2, external interface 250 is connected to physiological monitoring device 260 and permits the portable test unit 200 to receive physiological data, such as heart rate, blood pressure and skin conductance, from a test-taker.

In an embodiment of the present invention, the portable test unit 200 is implemented on a portable computer. By way of illustration and not as a limitation, the portable computer may be a Palm-based or other PDA, a handheld computer, a smart phone, or any similar or related handheld computing device that may be adapted to perform the functions of portable test unit 200.

The analysis of the data is performed automatically on the portable test unit, and the data may be transferred to another machine for more detailed analyses. Likewise, scores, performance level, and recommendations may be displayed on the testing machine, or on another computer, either on location or in a remote area.

The portable computer allows cognitive tests to be presented very quickly, providing a fast and accurate read out of a test-taker's processing abilities at that moment. In one embodiment of the present invention, each test assesses a very specific type of processing. Multiple assessments for multiple tasks may be presented and the test-taker may chose which sub-test to take. After each test, the user sees his or her mean RTs, mean variance in RT, and ER compared to norms. In a particular embodiment, an auditory version of the test is given, and records responses made verbally or by pressing a selection device, such as a bulb. The audio test may be given through an earphone, such as the sort commonly employed with cell phones.

In another embodiment of the present invention, the tests may be performed on wireless PDAs, which may be used to send the data immediately to a central site so that a supervisor can assess operational fitness while test takers are in the field. This system may run via XML, and which may function with any portable device that features a web browser, including cell phones or head-mounted devices.

Referring again to FIG. 2, both physiological data and cognitive performance are assessed by a “cognitive focus” test set. A cognitive focus test is provided that has components that will tax executive function, such as cognitive switching, working memory, and attention abilities. Thus, a single behavioral measure is obtained that will tap a set of processes that together are crucial for performance in a wide range of tasks. The cognitive focus test will require the test-taker to be vigilant for particular targets, and flexible in categorizing the targets. Stimuli may be auditory, such as audio information delivered to a discrete earplug connected to audio output 210. Examples of stimuli are letters of the alphabet that are named aloud, one at a time. Stimuli will vary according to the letter identity, pitch, volume and voice. In one embodiment test-takers will be required to respond each time a stimulus has at least two features, such as the same letter name read in the same voice, regardless of pitch and volume, or the same pitch and voice regardless of letter name or volume, that are identical to those of a stimulus that appeared one, two, or three trials previously. While this test is described herein with some specificity this is not meant as a limitation. Other embodiments of this type of test are well within the scope of the present invention.

Specific physiological measurements may be taken by, for example and not limited to, an ImagiProbe™ device produced by the Imagiworks (San Mateo, Calif.) to record heart rate, a portable 6371T from Omron Healthcare, Inc. (Vernon Hills, Ill.) to record blood pressure, and a specially adapted portable SCR device (the AT64, Autogenics, Wood Dale, Ill.) to record skin conductance.

The test results can be used by the workers themselves, either alone or as part of a team, or by a supervisor. For example, scores of the test may be accompanied by recommendations that workers be warned to pay additional attention and take extra care, or take a break, consume food or caffeine, or even take a nap.

An exemplary embodiment of the present invention uses portable test unit in a military setting. Military teams may be composed rapidly in response to an unexpected crisis. Thus, members not only may be pressed for time to prepare, but also may be fatigued and anxious. Moreover, because team members may be working together for the first time, their interactions may require extra effort. In addition, the setting of the assignment itself may be novel, requiring additional study and orientation to adjust to local culture and mores. Indeed, some team members may need to learn new skills on-the-fly, and employ them shortly after learning. All of these factors conspire to make individual performance, and that of the team as a whole, particularly vulnerable to cognitive impairment (such as is caused by fatigue). Thus, it is crucial to assess cognitive ability at regular intervals from the moment when a team is composed to when it completes its mission. The present invention uses cognitive and physiological measures to predict the task readiness of individuals and teams. Such methodologies and techniques are increasingly needed in the military, as well as many other areas.

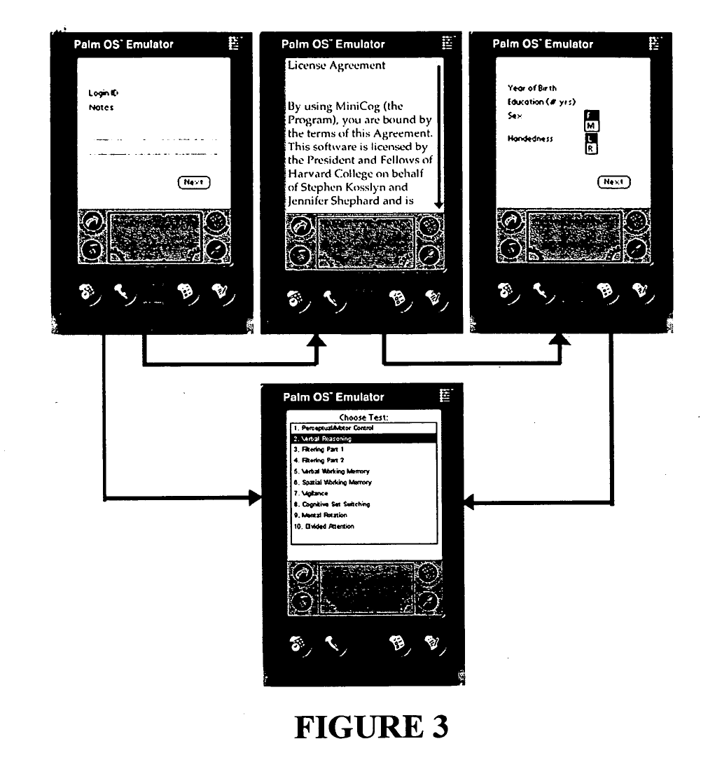

FIG. 3 illustrates the initial screens a user may see upon logging into the program, herein labeled “MiniCog,” on a portable device. As in many programs, the users receive the license agreement and inputs demographic data only upon the occasion of their first login. If the same ID is used with the same portable machine for additional testing sessions, the user will proceed immediately to the list of tests upon logging in.

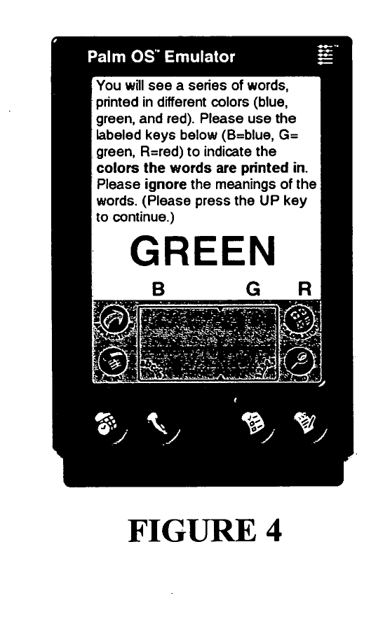

FIG. 4 illustrates a sample set of instructions for a test that may be displayed to the user once the test function has been selected according to an embodiment of the present invention. This and other tests are used to assess the cognitive abilities of the test-taker.

In one embodiment, the tests are scripted using an off-the-shelf HMTL editor, for instance Macromedia Dreamweaver or Netscape Composer, and then converted to a Palm OS compatible format using the compiler of the present invention. The operating systems noted above are not meant as a limitation. It is anticipated in the present invention the compiler may compile application programs for a variety of operating systems, not just for the Palm OS. As noted above, one element of the present invention is the software that allows new tests to be scripted and compiled based upon the tasks for which test takers will be evaluated. The tests may comprise both text and picture (jpg, gif, or png) stimulus files, present stimuli for specific amounts of time with a variable inter-stimulus delay, give auditory feedback after incorrect responses to practice trials, and record responses and response times from multiple key selection choices. The responses and RTs may be stored, along with a user ID and basic demographics (year of birth, years of education, gender, handedness, and notes) as well as a time-and-date stamp. In a particular embodiment, test-takers may be been given a password in order to be able to display their scores, which may be viewed immediately after each test is taken.

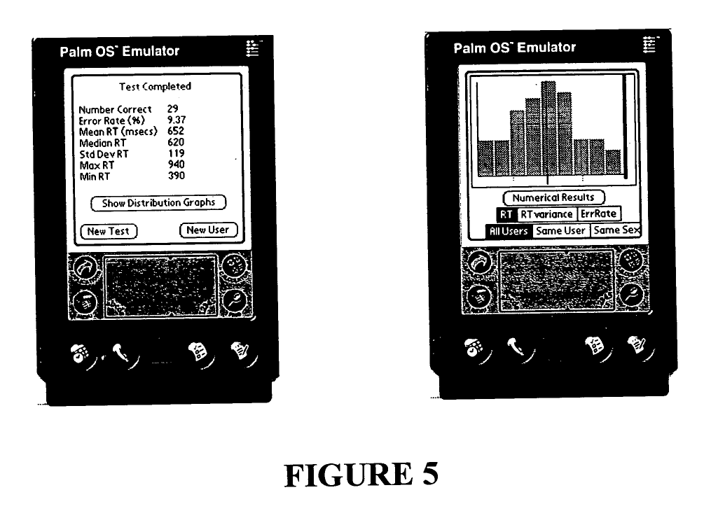

FIG. 5 illustrates a display of test results according to an embodiment of the present invention. As illustrated in FIG. 5, scores are presented numerically and as histograms. The histograms may compare current errors, response times, and response time variance to normative data for all users, for all users of the same gender as the current user, and for the current user.

In one embodiment, complete data sets, with user information and responses and response times for each trial of each run of each task, may be stored on the portable test device for later upload to a desktop computer where more detailed analyses can be performed.

Specific tests may differ in the instructions, which are presented in initial screens, stimuli, and the number of response keys used. The test may be designed in such a way that memorizing the stimuli does not give one an advantage in responding, and stimuli will be presented in different random orders each time the task is performed, so that the order of answers cannot be memorized. Thus, even if test-takers encounter the same items frequently within a short period of repeated testing, they will not be able to learn the correct responses by rote. In one embodiment, there is also an option to load in more stimuli than are needed, and stimuli are selected from this pool randomly each time the test is performed (and then removed from the pool until all the stimuli have been used), thus minimizing the number of times any given stimulus is repeated.

The test-taker may be told of the results of the test, or the results may be kept confidential. Predictions regarding the test-taker's performance on other tasks, and possible precautionary measures to implement, may be made available to the test-taker or to the supervisor, or both. A test giver may prohibit the test-taker from operating another device, or the prohibition may be automatic. For example, a truck driver may be required to take the test prior to being able to start the truck engine. Without proper taking of the test and comparing favorably to the norms of a population of truck drivers, the truck may not start, or an automatic warning may be given that operation of the vehicle must cease at a predetermined point. Other recommendations may also be triggered by the test results such as “you need more sleep” or other warnings. Thereafter a follow-up test may be taken again within a certain time frame.

The apparatus of the present invention may also comprise timing mechanisms whereby a user who does not pass the test after one or two tries, for example, may not be permitted to take the test for a predetermined interval to insure that rest is achieved or other prophylactic measures are taken.

In still another embodiment of the present invention, the test combines behavioral measures with physiological measurement, an auditory component, a visual component, a reflex component and combinations thereof. Any of the numerous physiological conditions that may be monitored may be part of the test, such as heart rate, body temperature, blood pressure, skin conductance, blood oxygen levels, or blood sugar level. These measurements may be used to assess adverse factors and competence to perform specific tasks, and also may help in distinguishing between adverse factors. For example, low body temperature may affect task performance in a similar way to alcohol levels, but may be compensated for by the test-taker more easily. In one particular embodiment, the entire test is given by audio components only, which allows the test-taker to perform certain other simple and routine tasks at the same time as taking the tests; this also allows visually impaired users to be tested.

In another embodiment of the present invention, a method for assessing the efficacy of cognitive processes is provided by inputting at least one test into a portable testing unit. The test is taken on the portable testing unit by a test-taker, whose performance is then evaluated to determine the efficacy level of at least one of the test taker's cognitive processes. The test-taker's performance may be limited to making selections and inputs on the portable testing unit, and the test itself comprises at least one visual or auditory component. By way of illustration and not as a limitation, a test may be selected from a list including a verbal working memory test, a spatial working memory test, a vigilance test, a filtering test, a divided attention test, a hand-eye coordination test, a verbal reasoning test, a spatial reasoning test, a cognitive set switching test, or a cognitive focus test.

In one embodiment of the present invention, the data and scores are stored on the handheld device.

In one embodiment of the present invention, the step of evaluating the test scores is at least in part performed on the handheld device. In another embodiment of the present invention, the step of evaluating the test scores is at least in part performed on a second device that receives the test score from the handheld device, either via a hard-wired connection or wirelessly.

In one embodiment of the present invention, the art of displaying the scores, performance levels, efficacy levels, and any recommendations is performed on the handheld device. In another embodiment of the present invention, the displaying of the efficacy levels, and any recommendations is performed on a second device that receives at least one of the test data, scores, performance levels, efficacy levels, or recommendations from the hand-held device.

In a particular embodiment of the present invention, the displaying of the scores, performance levels, efficacy levels, and recommendations requires the input of an authorization code.

In another embodiment of the present invention, the test data, scores, performance levels, efficacy levels, and recommendations may be associated with a time-and-date stamp, or personal data of the test taker, such as year of birth, gender, years of education, handedness, notes inputted by the test-taker, and reference number.

Nine tests are currently presented in the present invention although this is not meant as a limitation. As other cognitive tests evolve, it is fully within the scope of the present invention to include these tests as well. Current tests assess attention (vigilance, divided attention, and filtering), motor control, verbal and spatial working memory, verbal and spatial problem-solving, and cognitive set switching. Initially, a user should practice the tests a number of times under “normal” conditions until the user reaches a baseline performance level, so that when the battery is administered under stressful conditions, the data are not confounded by practice effects that compete with the effects of stress or other factors of interest.

Attention

Attention is the selective aspect of information processing, where processing of some information is facilitated while processing of other information is inhibited. If a person is unable to allocate attention appropriately during perception, he or she will not perform well. The present invention assesses three types of attention: vigilance, filtering, and divided attention.

Vigilance. Vigilance is the ability to concentrate and wait for a specific event over a sustained period of time. A series of small geometric shapes is presented one at a time on the screen. The set of shapes contains one target (a particular parallelogram) and five distracters. Because targets are presented occasionally and randomly, the test-taker must maintain concentration to detect them.

Filtering. Filtering is the ability to focus on what is important and ignore what is irrelevant to the task at hand. In the present invention color names appear on the screen of the PDA, one at a time, and the test taker is required to press the labeled button corresponding to the color of the ink while ignoring the meaning of the word. A black-and-white version has also been implemented, which requires users to name the number of digits on the screen while ignoring the meaning of the digits (e.g., they might see five ‘4’s, and must respond “five,” not “four”).

Divided. Divided attention requires one to focus on two unrelated types of stimuli or stimulus features. In the present invention, a series of four geometric shapes (circles, triangles, squares, and stars) in four different colors or shades (depending on whether the color or monochromatic version is used) are presented on the screen, one at a time. Test-takers are required to press one button if the shape is a triangle or is red (or dark grey, in the monochromatic version) and another button if the shape is a circle or blue (light grey); no triangles are blue/light grey and no circles are red/dark grey, to prevent response competition. In another version, users must keep track of both visual (objects) and auditory (pitches) information at the same time.

Working Memory

Information often must be “held in mind” as one manipulates it in some fashion, which requires working memory. Difficulty in using working memory interferes with thinking and problem solving, and most people who suffer from a working memory deficit are unable to think through and diagnose the cause of their difficulty because of the nature of the problem itself. A working memory test requires participants to see a series of stimuli and respond when a stimulus appears that had also appeared two (or, in more challenging versions, three) trials earlier in the sequence (this is called the “n-back” test). Two versions of this test have been implemented in the present invention.

Verbal working memory. A verbal version of this test requires the test-taker to keep track of a sequence of digits, keeping each in mind and deciding whether the digit seen two earlier in the sequence is the same as the one currently presented.

Spatial working memory. A spatial version of this test requires the test-taker to keep track of a sequence of locations, each indicated by the same digit. Four different locations are specified, and the test-taker must keep each in mind and decide whether the location seen two earlier in the sequence is the same as the one currently indicated.

Problem-Solving

To assess disruptions of very “high order” cognitive abilities, the present invention administers three tests, which require different types of problem solving and reasoning.

Verbal problems. The classic three-term series reasoning problems, such as “Kate is not as young as Sam; Kate is less old than Thomas—True or false: Sam is not oldest,” are administered. Test-takers indicate their responses by pressing one button for “true” and another for “false”.

Spatial problems. A test associated with mental rotation problems is also administered. Pairs of stimuli are presented, with one member of the pair rotated relative to the other. Test-takers are required to try to “mentally align” one form so that it is at the same angle as another, and then to decide whether the two are identical or mirror-reversals. The angle of disparity between the members of the pair varies, for both “same” and “mirror” pairs.

Cognitive set switching. These tests require participants to sort stimuli (typically simple figures or letters) according to different criteria. The stimuli are presented on a deck of cards, and the figures or letters on each card can be classified into more than one category (e.g., a given card might have three purple squares; this card could be categorized according to number, color, or shape). Participants begin to sort cards into different piles based on one of three possible criteria (shape, color, number). The test-takers receive feedback after each trial (they are simply told whether they have sorted that particular stimulus correctly), and from this feedback must learn the appropriate sort criterion (predetermined by the administrator). Once they have learned the criterion (learning is defined as correctly responding to a specific number of trials in a row), the sort criterion is arbitrarily switched (e.g., from number to shape), and the process begins again. The key measure is how many trials a participant requires before he or she discovers the new criterion. The present invention presents a version in which each trial presents a series of four letters (e.g., T H t H), and participants must decide which letter does not belong in the series (in this case, the lowercase ‘t’). There are two stimulus criteria (letter identity and capitalization), only one of which applies to any given trial. Two, three, four or five (randomly determined) trials in a row have the same criterion, and then the criterion switches.

Perceptual and Motor Control

It is important to discover whether changes in response times or errors on the tests are due simply to a slower motor response rather than to cognitive deficits. For instance, hunger, cold, or gloves could affect the ability of a user to move his/her fingers quickly and accurately across the rather small and closely spaced keys on a PDA. Therefore a task is included where a stimulus (a small oval) appears above randomly selected keys on the PDA. Users must respond by pressing the corresponding key as quickly as possible.

As noted above, these tests are not meant as a limitation. For example, tests of the present invention may also combine various features. For instance, a “Cognitive Focus” test can also tap aspects of both vigilance and verbal and spatial working memory together.

A system and method for on-site cognitive efficacy assessment have been described. It will be understood by those skilled in the art that the present invention may be embodied in other specific forms without departing from the scope of the invention disclosed and that the examples and embodiments described herein are in all respects illustrative and not restrictive. Those skilled in the art of the present invention will recognize that other embodiments using the concepts described herein are also possible. It is anticipated, for example, that the test device of the present invention may comprise a connection (wired or wireless) to equipment associated with an activity such that the equipment may not be used if test results are below a normative level. Thus the present invention will find particular use in operation of potentially dangerous equipment such as in construction, automotive and aviation fields. Additionally, other platforms may be used to administer the tests of the present invention such as cellular telephones or other portable computing devices such as laptop computers and the like. Further, any reference to claim elements in the singular, for example, using the articles “a,” “an,” or “the” is not to be construed as limiting the element to the singular.

1. A method for assessing relative levels of cognitive performance comprising:
presenting to a test-taker a test object indicative of a cognitive function;
receiving a response to the test object from the test-taker;
calculating a score from the response;
evaluating the score against normative data to determine a performance level of the cognitive function of the test-taker; and
displaying the score and the performance level.

2. The method of claim 1, wherein receiving a response to the test object from the test-taker comprises receiving an answer and wherein the cognitive function is measured by response time and error rate.

3. The method of claim 1, wherein evaluating the score against normative data to determine a performance level of the cognitive function of the test-taker comprises evaluating the score against normative data to determine a performance level of the cognitive function of the test-taker relating to at least one of attention, working memory, problem-solving, cognitive-set-switching, perceptual control, motor control, and cognitive focus.

4. The method of claim 1, wherein presenting to a test-taker a test object indicative of a cognitive function comprises:
presenting to a test-taker a test object indicative of a cognitive function on a portable testing unit; and
wherein the method further comprises storing on the portable testing unit at least one of the response, the score and the performance level.

5. The method of claim 4, wherein evaluating the score comprises evaluating the score on the portable testing unit.

6. The method of claim 4, wherein evaluating the score comprises evaluating the score on a second device that receives the score from the portable testing unit.

7. The method of claim 4, wherein displaying at least one of the score and the performance level comprises displaying on the portable testing unit.

8. The method of claim 4 wherein displaying at least one of the score and the performance level comprises displaying on a second device that receives at least one of the score and the performance level from the portable testing unit.

9. The method of claim 1, further comprising associating a time-and-date stamp with at least one of the response, the score and the performance level.

10. The method of claim 1, further comprising associating personal data of the test-taker with at least one of the response, the score and the performance level.

11. The method of claim 10, wherein the personal data comprises at least one of year of birth, gender, handedness, years of education, notes, and reference number.

12. The method of claim 1, wherein displaying at least one of the score and the performance level comprises inputting an authorization code.

13. A method for determining impairment in brain function in relation to specific adverse factors comprising:
establishing a threshold for cognitive performance for an activity;
administering to a test-taker a test on a portable testing unit, wherein the test is adapted to measure impairment due to adverse factors and to produce a score; and
determining an efficacy level of the test-taker;
wherein the determining of the efficacy level comprises comparing the score to the threshold for the activity

14. The method of claim 13, wherein the method further comprises suggesting a precautionary action related to the activity if the threshold is not met.

15. The method of claim 13, wherein the adverse factors comprises at least one of sleep deprivation, drugs, alcohol, sensory distractions, physiological conditions, and psychological conditions.

16. The method of claim 13, wherein the test-taker is a group of individuals, each taking the test thereby establishing the threshold.

17. The method of claim 13, wherein the portable testing unit is a portable computer.

18. The method of claim 13, wherein the test further comprises at least one of a physiological measurement, an auditory component, a visual component, and a reflex component.

19. A method for qualifying a user for an activity based upon assessing relative levels of cognitive performance comprising:
establishing a threshold of cognitive functioning necessary for performing a particular activity;
presenting to a user a test object indicative of a cognitive function associated with an activity;
receiving a response to the test object from the user;
calculating a score from the response;
evaluating the score against the threshold to determine a performance level of the cognitive function of the user;
displaying the score and the performance level of the user; and
displaying a recommendation to the user regarding the activity based upon the score and performance level.

20. The method of claim 19 further comprising preventing the user from engaging in the activity if the score and performance level are below a normative level for the activity.

21. The method of claim 19, wherein the threshold for performance is based upon population norms

22. The method of claim 19, wherein the threshold for performance is based upon the user's own prior scores.

23. The method of claim 20 further comprising preventing the user from engaging in the activity if the score and performance level are below a threshold for the activity.

24. An apparatus for qualifying a user for an activity based upon assessing relative levels of cognitive performance comprising:
means for presenting to a user a test object indicative of a cognitive function associated with an activity;
means for receiving a response to the test object from the user;
means for calculating a score from the response;
means for evaluating the score against normative data for the activity stored within a data store to determine a performance level of the cognitive function of the user; and
a display for presenting the score and the performance level of the user and for displaying a recommendation to the user regarding the activity based upon the score and performance level.

25. The apparatus of claim 24 further comprising a connection between the means for evaluating the score against normative data for the activity and equipment used for the activity; and whereby use of the equipment is denied if the performance level of the cognitive function of the user is below the normative data.

26. The apparatus of claim 24 wherein:
the means for presenting to a user a test object indicative of a cognitive function associated with an activity;
the means for receiving a response to the test object from the user;
the means for calculating a score from the response;
the means for evaluating the score against normative data for the activity stored within a data store to determine a performance level of the cognitive function of the user; and
the display comprise a cellular telephone.

27. The apparatus of claim 24 wherein:
the means for presenting to a user a test object indicative of a cognitive function associated with an activity;
the means for receiving a response to the test object from the user; the means for calculating a score from the response;
the means for evaluating the score against normative data for the activity stored within a data store to determine a performance level of the cognitive function of the user; and
the display comprise a Personal digital assistant (PDA)

28. The apparatus of claim 24 wherein:
the means for presenting to a user a test object indicative of a cognitive function associated with an activity;
the means for receiving a response to the test object from the user;
the means for calculating a score from the response;
the means for evaluating the score against normative data for the activity stored within a data store to determine a performance level of the cognitive function of the user; and
the display comprises a laptop computer.
